(19) United States
(12) Patent Application Publication
(10) Pub. No.: US 2019/0287678 A1
(43) Pub. Date: Sep. 19, 2019

(54) Monitoring Device, System, and Method for Incontinence Sensor Pad and Transmitter

(71) Applicant: G2i Incorporated, Palo Alto, CA (US)

(72) Inventors: Thomas Reed Stevens, Palo Alto, CA (US); Ivan J. Goering, Palo Alto, CA (US)

(21) Appl. No.: 16/463,032

(22) PCT Filed: Nov. 22, 2017
(86) PCT No.: PCT/US2017/063042

§ 371 Date: May 22, 2019

Related U.S. Application Data
(60) Provisional application No. 62/425,890, filed on Nov. 23, 2016.

(51) Int. Cl. G16H 40/67; A61B 5/00; A61B 5/20; A61F 13/42

(52) U.S. Cl. CPC G16H 40/67; A61B 5/746; A61B 5/202; A61F 2013/424; A61F 13/42; A61B 2562/029; A61B 5/0022

(57) ABSTRACT

A monitoring device is disclosed. The monitoring device includes a sensor configured to determine moisture data associated with moisture in a pad; and a transmitter configured to connect to the sensor and transmit the moisture data to a computer system comprising one or more processors.

### CROSS-REFERENCE TO RELATED APPLICATIONS

This application claims priority to U.S. Provisional Patent Application Ser. No. 62/425,890, filed Nov. 23, 2016, the entire disclosure of which is hereby expressly incorporated by reference herein.

### COPYRIGHT NOTIFICATION

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

### 1. Field of the Disclosure

The present disclosure relates generally to a monitoring device and system. More particularly, the present disclosure relates to a sensor pad and transmitter for tracking patient incontinence.

### 2. Description of the Related Art

Incontinence in patient care environment is a growing problem in patient care and home care of elderly patients. Urinary incontinence is the involuntary leakage of urine. Many patients have the inability to hold urine in their bladder because voluntary control over the urinary sphincter is either lost or weakened. Urinary incontinence is a much more common problem than most people realize.

It is common for nursing homes and hospitals to lack the staff and financial resources to provide residents with sufficiently frequent toileting assistance (including prompted voiding). Use of special undergarments and absorbent pads or catheterization is the usual practice.

Urinary incontinence (UI) and fecal incontinence (FI) are commonly encountered in nursing home residents and are associated with significant morbidity and utilization of health care resources. Urinary incontinence has been estimated to affect between 50% and 65% of nursing home residents, and a majority of these residents also have FI. UI is also prevalent in the at-home aging population and is a leading factor in senior isolation and eventual institutionalization in a care facility.

There are several key activities of daily living (ADL) that are indicative of quality of life and safety in an aging population including: toileting, sleep, medication, and nutrition. Incontinence is a critical ADL deficit that negatively impacts all aspects of autonomy, health, and overall well-being. It is a leading cause of seniors' loss of independence and requiring professional care. The demand for improved incontinence solutions exist, in ever increasing levels of severity, at every stage in elder care from family caregiving through to acute care hospitalization, with the highest utilization rates occurring in long-term living facilities. Sleep quality is another key indicator that augments and inter-relates with incontinence.

Elderly people constitute a large and growing portion of the world's population. Many of them are physically and mentally vulnerable and need continuous support for their health and well-being. There is a growing trend that these elderly people are placed in an ambient assisted living environment (AAL) with an aim to receive better care and support. However, much less attention has been directed toward understanding incontinence needs of elderly people, which is an important factor relevant to their physical and mental health and joyful living.

One in three adult women live with some level of urinary incontinence. Nearly 40% (19 million) of all seniors and over 60% (15 million) of female seniors live with incontinence, with increasing prevalence and severity as age increases. Suboptimal incontinence care leads to degenerative skin health, an increased risk of falls as patients unsuccessfully attempt to self-toilet, and critical declines in mental health. As a result, it is the leading cause of senior isolation and institutionalization. Clinical nurses and the research community agree that there is clear correlation between incontinence and pressure ulcers and urinary tract infections (UTIs). UTIs and pressure wounds are directly linked to increased negative outcomes.

The cost to treat pressure ulcers can be very expensive and is estimated between $9.1-11.6 billion per year, affecting over 2.5 million patients. Approximately 60,000 people die each year as a direct result of a pressure ulcer. Keeping the skin free from exposure to urine and stool is very important in treating pressure ulcers and bedsores. Similarly, UTIs are rampant as well, as a result of over-catheterization, totaling over $340 million per year and with at least 13,000 deaths a year are associated with UTIs. Increased costs and negative outcomes with UTIs are likely as the patient population grows older. The known solutions that demonstrate improvement in these costs and outcomes are needed.

For enterprise businesses, incontinence is a significant issue. For caregivers, such as acute care hospitals, incontinence is a contributor to revenue loss and a key source of family dissatisfaction with institutional providers. Nearly $4 billion is spent on adult non-woven absorbency products in the US ($9 billion globally), and the segment is growing as the Baby Boomers continue to age and live longer than their predecessors.

It is known that the complications of urinary incontinence are increasingly and rapidly expanding as the world's population is aging longer with each New Year. Many elderly people encounter skin problems, but an elderly person with urinary incontinence is even more likely to have skin sores, rashes, and infections because the skin is wet or damp. This is bad for wound healing and also promotes fungal infections. Urinary tract infections are a significant risk, and long-term use of urinary catheters also significantly increases the risk of infection.

The problem has been addressed in part by providing pads that are manually replaced when the nurse is visiting a room. The amount of times a product needs changed depends in part on how absorbent the pad, diaper, or pull-up is and the severity of the incontinence. Generally, it is best to change a product as soon as soiling occurs. This will reduce the risk of skin breakdown and infections caused by a lack of air flow, moist conditions, and long exposure to urine and fecal matter.

With each change, it is important to thoroughly clean the diaper area to reduce infections. After changing, it is important to properly dispose of soiled incontinence products.

Disposable briefs are more commonly known as adult diapers. Adult diapers are often used for heavy incontinence, nighttime wetting, and those who need help getting to the bathroom.

Therefore, there is a need to provide methods an apparatus for improved incontinence sensing. Thus, there remains a considerable need for pads with improved incontinence sensing and systems that can quickly and accurately address a patient with a wet pad.

### SUMMARY OF THE INVENTION

There currently exists a need for sensor pad systems for managing incontinence adapted to new patient care facilities. Systems for coupling complex sensor pads with software tracking systems and monitoring systems are also needed. In care facilities today, only manual systems exist for the management and maintenance of patient bedding. Many care facilities have no way to determine, monitor, and schedule service and visits based on the real time needs of the patient. Often patients are left in their own urine and feces for extended periods of time, causing many health problems. This leads to increased demands for alternative, pad based incontinence solutions.

There currently exists a need for incontinence protection having improved in the effectiveness at drawing moisture away from the body and keeping odors at bay. In addition, a need exists for maintaining skin health by keeping the perineal area dry and making sure the smell of urine or feces doesn't become noticeable to others are essential to maintaining quality of life —both physical and emotional.

Specifics for incontinence are usually measured by total exposure time (per void and cumulative), and the number of long-term acute care (long-term acute care) people that have used pads over the last ten years. There is a huge growth in catheterization, which in turn has led to a huge rise in catheter related urinary tract infections. Prior to our solution, nobody has actually been able to determine this metric. So currently there is lots of agreement that a correlation between exposure time and negative outcomes exists, but nobody knows what the actual relationship is and where the tipping point into a risk factor is.

In accordance with an embodiment of the present disclosure, a monitoring device includes a sensor configured to determine moisture data associated with moisture in a pad; and a transmitter configured to connect to the sensor and transmit the moisture data to a computer system comprising one or more processors.

In one configuration, the moisture data is associated with at least one of the following: a moisture level in the pad, a moisture location in the pad, a moisture type in the pad, or any combination thereof. In another configuration, the sensor is attached to an interior of a garment. In yet another configuration, the sensor and the transmitter are removably connectable. In one configuration, the monitoring device includes a sensor pad having a top layer formed of a flexible material; an integrated sensor layer including the sensor; and an absorption layer disposed between the top layer and the integrated sensor layer. In another configuration, the top layer wicks a fluid across an area of the top layer. In yet another configuration, the absorption layer absorbs and stores a fluid. In one configuration, the monitoring device includes a powder disposed within the absorption layer, wherein the powder forms into a gel as the absorption layer absorbs a fluid. In another configuration, the integrated sensor layer includes a first sensor arranged in an interior central detection zone; and a second sensor arranged in a perimeter detection zone. In yet another configuration, the integrated sensor layer includes a first sensor arranged in a first spiral configuration; a second sensor arranged in a second spiral configuration; and a third sensor arranged in a rectangular configuration outside the first spiral configuration and the second spiral configuration. In one configuration, the integrated sensor layer is waterproof. In another configuration, a portion of the integrated sensor layer includes a perforation. In yet another configuration, the transmitter includes a top portion; a bottom portion; and a connection portion that movably connects the top portion and the bottom portion between an open position and a closed position. In one configuration, with the connection portion in the closed position, a cavity is formed between the top portion and the bottom portion. In another configuration, the transmitter includes an electrical connector. In yet another configuration, with the connection portion in the closed position, a portion of the sensor is received through the cavity of the transmitter and contacts the electrical connector to connect the transmitter and the sensor. In one configuration, the sensor is energized with a ground signal, and wherein, with moisture on the integrated sensor layer, a circuit is formed with the transmitter. In another configuration, the transmitter includes at least one processor programmed or configured to recognize and transmit characteristics of electric signals in the circuit. In yet another configuration, the transmitter includes at least one processor programmed or configured to monitor, with the computer system, the moisture information and send an alert to a user based on the moisture information. In one configuration, the processor is programmed or configured to recognize and transmit a capacitance of the sensor. In another configuration, the processor is programmed or configured to recognize and transmit an inductance of the sensor. In yet another configuration, the processor is programmed or configured to recognize and transmit a temperature of the sensor. In one configuration, the processor is programmed or configured to recognize and transmit an impedance of the sensor. In another configuration, the processor is programmed or configured to recognize and transmit characteristics of the moisture information. In yet another configuration, the transmitter is re-usable and the sensor is disposable.

In accordance with another embodiment of the present disclosure, a monitoring system includes a sensor configured to determine moisture data associated with moisture in a pad; a transmitter configured to connect to the sensor and transmit the moisture data to a computer system comprising one or more processors; and the computer system configured to receive the moisture data from the transmitter to determine when a patient needs attention and send an alert to a user based on the moisture data. For example, a monitoring system includes determining, with a sensor, moisture data associated with moisture in a pad; and transmitting, with a transmitter connected to the sensor, the moisture data to a computer system comprising one or more processors.

In accordance with another embodiment of the present disclosure, a computer system includes one or more processors programmed or configured to: receive moisture data associated with moisture in a pad; determine based on the moisture data when a patient needs attention; and sending an alert to a user based on the moisture data.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a diagram showing a Patient Incontinence Monitoring System in accordance with an embodiment of the present invention.

FIG. 2A is a perspective view of a transmitter in an open position in accordance with an embodiment of the present invention.

FIG. 2B is a perspective view of the transmitter in a closed position, with male connectors and female connectors, in accordance with an embodiment of the present invention.

FIG. 2C is a front elevation view of the transmitter in a closed position in accordance with an embodiment of the present invention.

FIG. 2D is a top elevation view of the transmitter in accordance with an embodiment of the present invention.

FIG. 2E is a rear elevation view of the transmitter in accordance with an embodiment of the present invention.

FIG. 2F is a side elevation view of the transmitter, with movable connecting pieces, in accordance with an embodiment of the present invention.

FIG. 2G is a perspective view of the transmitter in an open position, with connecting pieces, in accordance with an embodiment of the present invention.

FIG. 3 is a block diagram of the internal transmitter in accordance with an embodiment of the present invention.

FIG. 3B is a diagram of a non-limiting embodiment of components of one or more devices of the present invention;

FIG. 4 is an exploded view of the sensor pad formed of three absorbent layers in accordance with an embodiment of the present invention.

FIG. 5 is an elevation view of a sensor pad in accordance with an embodiment of the present invention.

FIG. 6 is a perspective view of a pad sensor coupled to a transmitter in accordance with an embodiment of the present invention.

FIG. 7A is a perspective view of a transmitter in an open position in accordance with another embodiment of the present invention.

FIG. 7B is a side elevation view of the transmitter in a closed position in accordance with another embodiment of the present invention.

FIG. 7C is a top elevation view of the transmitter in accordance with another embodiment of the present invention.

FIG. 8A is a perspective view of a transmitter in an open position in accordance with another embodiment of the present invention.

FIG. 8B is a side elevation view of the transmitter in a closed position in accordance with another embodiment of the present invention.

FIG. 8C is a top elevation view of the transmitter in accordance with another embodiment of the present invention.

FIG. 9A is a perspective view of a transmitter in an open position in accordance with another embodiment of the present invention.

FIG. 9B is a side elevation view of the transmitter in accordance with another embodiment of the present invention.

FIG. 9C is a side elevation view of the transmitter in accordance with another embodiment of the present invention.

FIG. 10 is a flowchart of a non-limiting embodiment of a process for monitoring a patient.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

The present disclosure provides a Patient Incontinence Monitoring System for electronically detecting the presence of moisture in a patient care or home care environment. It can send a detection of moisture across a network to a third-party device 25 (e.g., a computer, a remote pad, a smartphone, a cloud) for enabling the remote collection and analysis of incontinence data. This detection can also be used by a third-party device 25, such as a monitoring system, to determine patterns and/or alert a caregiver associated with an incontinence event.

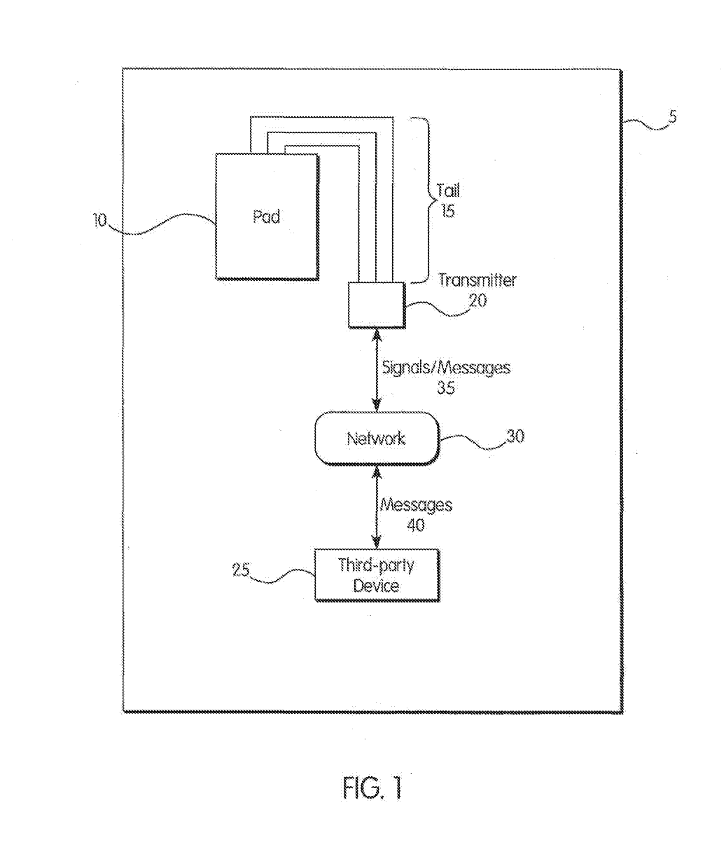

With reference to FIG. 1, a Patient Incontinence Monitoring System 5 includes a multi-layer sensor pad 10, a tail 15, and a transmitter 20. The tail 15 extends from the sensor 10 and is operative to connect the transmitter 20 to the multi-layer location-based sensor pad 10 to read signals 35 from pad 10 and transmits pad data in the form of signals and/or messages 35 across a network 30 to a third-party device 25, such as internet service, cloud service, hosted or standalone computer, iPad, smartphone, database, or other transmitter/repeater. The third-party device 25 uses the pad data to determine that moisture is present and begins to track and/or monitor the moisture on the multi-layer location-based sensor pad 10. The third-party device can be a specially programmed computer intended to utilize the sensor pad data of multi-patient environments.

The tail 15 is integrated with the pad itself. In a preferred embodiment, the tail 15 is formed as part of the sensor pad 10, created within the manufacturing process of the pad 10. The sensors of the pad are printed onto a flexible material and then joined with the other layers of the sensor pad 10. In an alternate embodiment, not shown, the sensors can be attached using an adhesive or some other material or compound to fasten the sensor. In a preferred embodiment, a unitary sensor is used to form the pad 10 and the tail 15. The tail forms an extension of the sensor from the body of the pad sensor and providing length and flexibility to reach and connect to the transmitter 20. The transmitter/tail interface provides a soft point of failure for the transmitter and pad combination to ‘fail’ in the instance of a fall or tripping hazard situation. In contrast to a hard flex circuit or some sort of materially strong connection between the pad and the transmitter that creates a fall hazard, the tail 15 is defined to easily and quickly tear or pull from the transmitter to avoid accidents such as falling. The flexible tail 15 is formed by perforating a part of the material that divides the tail portion from the body portion of the sensor pad. For example, a line forming a path between the sensors of the tail and the sensors of the body. When the perforation is detached a flexible tail is formed, extending from the sensor pad and manipulatably flexible for connecting to a transmitter 20. The tail can also be easily removed after the pad 10 has been consumed. The tail 15 easily torn from the pad body while the tail 15 is still connected to the transmitter 20. The pad body can be easily disposed of, leaving the transmitter which can be removed after the old tail is removed and disposed.

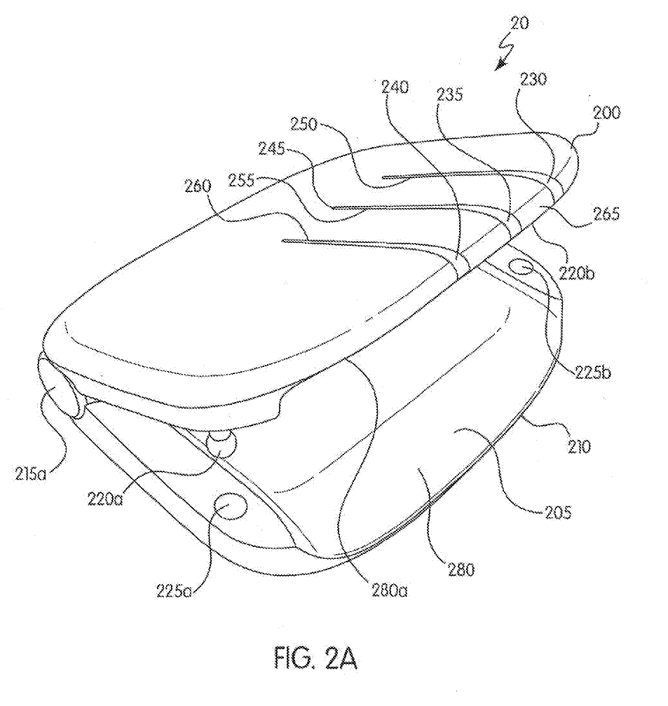

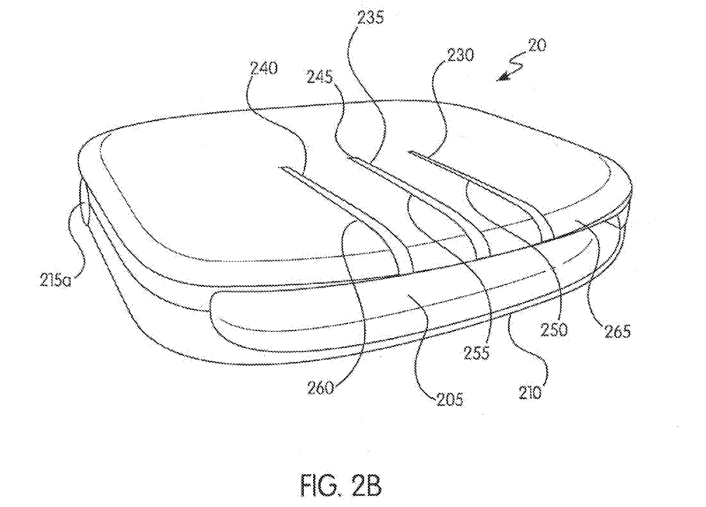

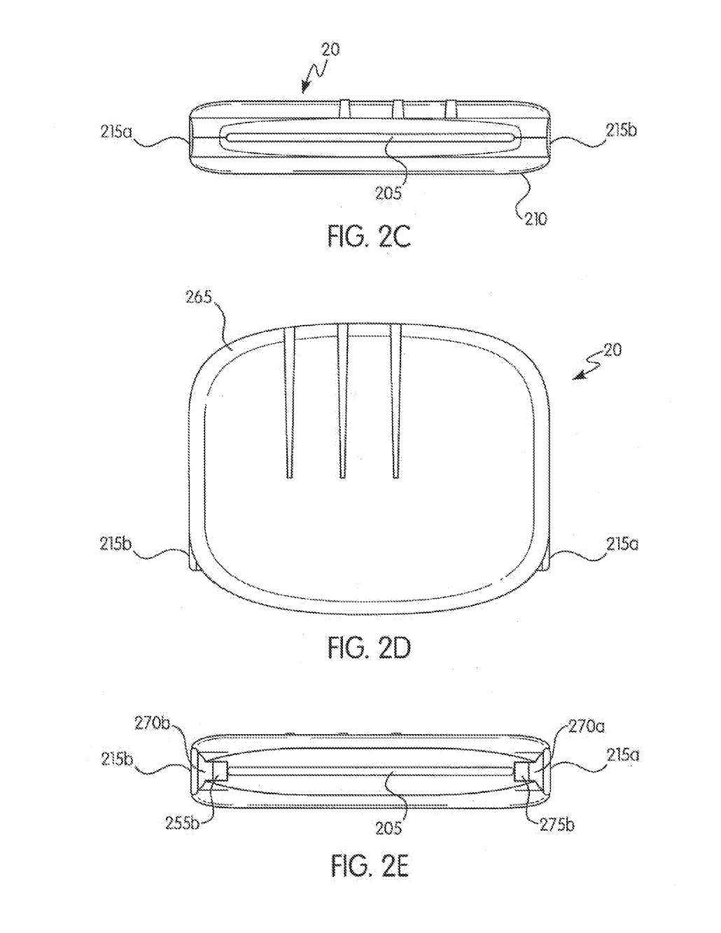

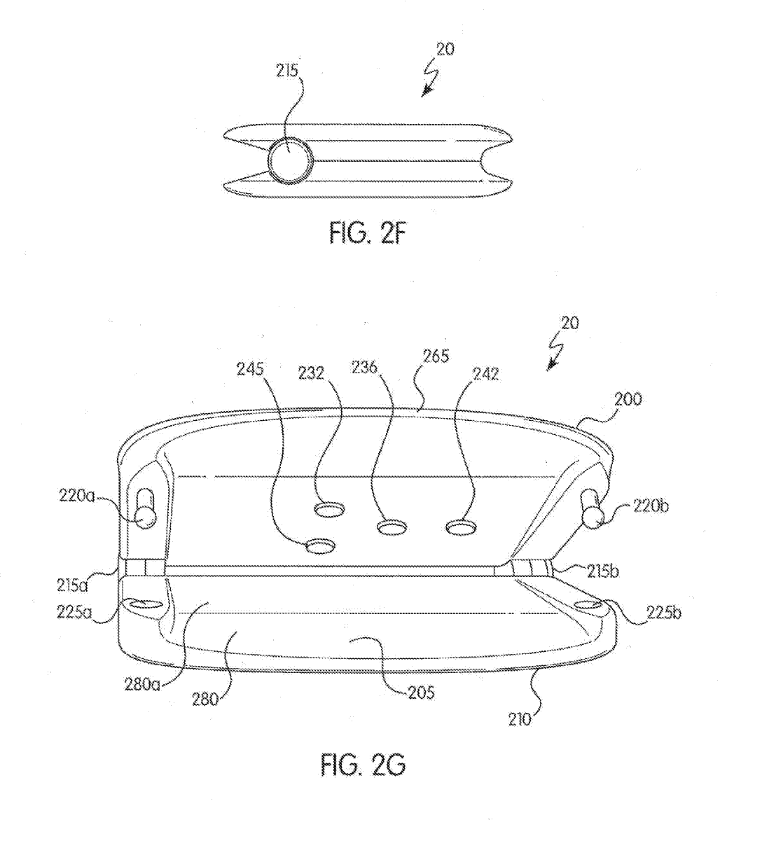

With reference to FIG. 2A, the transmitter 20 has a smoothed, rounded top panel 200 and a smooth, rounded bottom panel 210 that are connectably secured together by a movable connecting piece 215a, 215b (shown in FIG. 2D), forming a clamshell with congruent joints on either end. Such a shape of the upper surface 200 increases ergonomics of the transmitter, its curvature following the natural curvature of the human hand, thereby enhancing grasping comfort. The movable connecting pieces 215a, 215b are inserted into circular openings 270a and 270b (shown in FIG. 2E) in the top panel 200 and mirrored circular openings formed in the bottom panel 210, the movable connecting pieces 215a, 215b form an axis of a rotation about the joint, where the panels move about the axis, to open and close, by rotating the panels 200 and 210, relative to each other. Both the top panel 200 and the bottom panel 210 have rounded edges and internally curved inside surfaces formed on the internal surfaces of panels 200, 210, that face each other, such that when the pieces are closed together a cavity 205 is formed (shown in FIG. 2B). The internally curved insides are stepped internally down such that an inner portion 280 is thinner than the outer portion. This design facilitates the receiving of a tail region 15 as shown in FIG. 1. Extending outward from the top panel 200 is a male connector 220a on one side and male connector 220b on the other side. Extending outward from bottom piece 210, and directly opposite the male connectors of top panel 200, are female openings 225a and 225b. The male connectors 220a, 220b form a locking connection between the panels 200 and 210 when they are inserted into the mating female openings 225a, 225b and received therein. However, one of ordinary skill would recognize that locking surfaces can be formed with other means, where the transmitter may be closed and secured.

In one embodiment, the transmitter is side hinged, and instead of the tail running through the middle of the transmitter and out the back under the hinge, the hinge is to one side of the tail and the transmitter clamps across it from the side.

With reference to FIGS. 2B-2F, the transmitter 20 of FIG. 1 is shown from various angles. Referring to FIG. 2B, the transmitter 20, as previously discussed, is closed by inserting male connectors 220a, 220b into the female connectors 225a, 225b, respectively (shown in FIG. 2A). The closing of the transmitter 20 will create the cavity 205 through the closed transmitter 20. The closing of the transmitter 20 and creation of the cavity 205 for receiving the tail 15 inserted through and connected to the transmitter 20 (discussed in more detail later). With reference to FIG. 2C, the transmitter 20 is shown from the front, and having an opening where the tail end can be inserted into the transmitter 20. With reference to FIG. 2D, the transmitter 20 is shown from the top such that the curved edges of the top piece 200 can be easily realized. In other words, the upper surface is curved more near its ends where the degree of curvature is increased, and more flat in the vicinity of its middle, where the radius of curvature is less. Such a shape of the surfaces of the panels, increases ergonomics of handling the transmitter, its curvature following the natural curvature of the human hand, thereby enhancing grasping comfort. FIG. 2E shows the smart transmitter from the back to show the back opening where the tail end may come out. FIG. 2F shows the transmitter 20 from the right such that the movable connecting pieces 215a, 215b can be seen along with the top piece 200 and the bottom piece 210 closed down.

As shown in FIGS. 2C and 2E, the cavity 205 is the same width throughout the transmitter 20 such that the tail 15 (not shown) can be inserted through at the same width. However, this is not meant to be construed in a limiting sense and the cavity 205 may be the same or different widths throughout the transmitter 20. Grooves or lines on the transmitter are used to provide a visual cue that the user correctly handles the alignment of the pad ribbon with the transmitter contacts. Such an inclination improves the ergonomics of the transmitter 20, especially in the open and close position where the lines of the smoothed top and bottom panels 200 and 210 are complimenting the internal cavity and path there through for connecting the tail of the sensor pad to the transmitter. The ergonomic design of the top and bottom provide stability for holding and positioning the tail therein, the rounded surfaces ergonomically easing the use of the transmitter. In one embodiment, the length of the first and second edge are 100 mm and 80 mm, a height of 20 mm, with a 30 degree opening in the back end of the transmitter, whereas the cavity having an opening of less than 20 mm in height when closed.

There is an LED indicator by the logo that flashes green when a pad is connected to indicate that the contacts have made contact with the pad. The LED will then flash red when moisture is detected on the pad providing a visual local indicator, and also when the transmitter has not been properly connected to a new pad (i.e., it will not ‘go green’ until it connects to a new dry pad).

Referring again to FIG. 2A, three connectors 230, 235, 240 are inserted into the top panel 200 of the transmitter 20. Each connector is inserted into a formed connector opening formed on the top surface such that when the connector running parallel from the top panel 200 down to a front edge 265 is inserted into the connector cavity the connector is flush with the top panel 200. As shown in FIG. 2A, the connectors 230, 235 and 240 are inserted into connector openings 250, 255 and 260, respectively, formed in the top surface. The connectors 230, 235, 240 are attached to the transmitter 20 and wired to the boards. For example, the connectors 230, 235, 240 can be glued or snapped onto the transmitter 20. Also shown in FIG. 2A, connector 245 sits internal to the transmitter 20 at the top of connector 235. The contacts are mounted to the circuit board and protrude from holes formed during injection molding. These contact pins are inside the clamshell with the tail going through the shell like a belt in a buckle. The three stripes on the exterior of the transmitter are a visual queue for quick functional alignment. The contacts are connected directly to the control board and provide the electrical charge as well through the coupling of the board and the sensor pad. The connectors can be formed of prongs on an internal surface. In one embodiment, at least two of the prongs hit grounding trace. They can create a short contact coupling used to communicate that the tail 15 is plugged into the transmitter 20 correctly. That connection is keeping the outer ring charged all the time. Because the things is on all the time, this drops the impedance level into the range that it is tuned for. When that happens, it wake up the processor.

With reference to FIG. 2G, the smart transmitter 20 is clamped onto the tail 15 aligning physical connectivity for the connectors 230, 235, 240, with the sensors, 30, 35, 40, on the tail 15. The connectors 230, 235, 240 on the transmitter 20 have conductive pins 232, 236, and 242 that are pressed against the electric sensors 30, 35, 40 and coupling with them to create the electrical connection. Through the connection, the smart transmitter 20 receives the moisture information from the sensors 30, 35, 40. The conductive pins 232, 236, and 242 can be blunt or machined as shown in FIG. 2G or alternatively, in another embodiment the conductive pins can have a sharp edge or point that can penetrate the sensors 30, 35, 40 to form a connection.

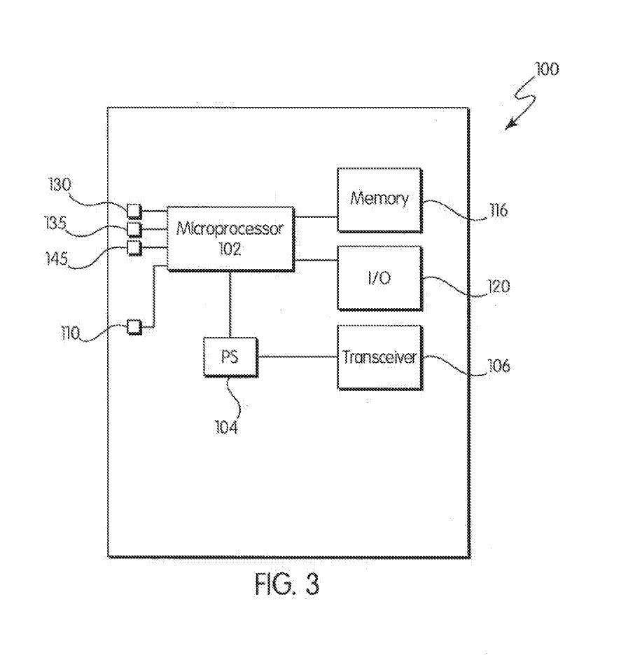

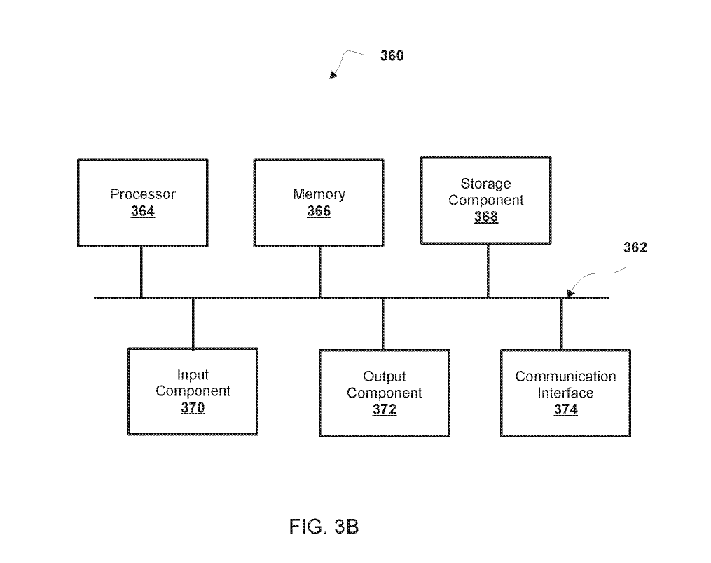

The transmitter 20 of FIG. 1 includes a board 100, shown in FIG. 3. The board 100 has a power supply 104, a microprocessor 102, a transceiver 106, internal connectors 130, 135, 145, memory 116 and input/output 120. The power supply 104 can be any conventional circuit for providing, controlling, converting, measuring, and/or detecting a voltage and/or current. One of skill in the art would understand other sources of power besides a battery could be used, such as traditional plug and socket or other adapter, a power outlet, green, or USB type connection, to provide electrical energy. The transmitter 20 includes a memory 116 for data and instruction storage 116. In an embodiment, information relating to the specific device platform (e.g., ID information, history) and/or patient information (name, age, moisture frequency, social security number) is stored on the board memory. The internal connectors 130, 135, 145 connect to the connectors 230, 235 and 240 (shown on FIGS. 2A and 2B) on the transmitter 20 and relay sensor information or messages to the microprocessor 102. The transceiver 106 receives the sensor information or messages which can then be transmitted to the network and then to a third-party device 25. The input/output 120 is coupled to the microprocessor 102 and allows for a user to input additional sensor data. According to a non-limiting embodiment or example, the microprocessor 102 is electrically coupled to the sensors 30, 35, 40 through the connectors 230, 235, 240. The transceiver transmits and receives signals wirelessly. Alternatively, the transceiver does not need to be wireless.

Referring now to FIG. 3B, FIG. 3B is a diagram of example components of a device 360. Device 360 may correspond to one or more devices of Patient Incontinence Monitoring System, one or more devices of a transmitter of at least FIGS. 2, 7, 8 and 9, and/or one or more devices (e.g., one or more devices of a system of) of power supply 104, microprocessor 102, transceiver 106, memory 116 and input/output 120. In some non-limiting embodiments, one or more devices of patient incontinence monitoring system, one or more devices of a patient incontinence monitoring database, and/or one or more devices (e.g., one or more devices of a system of) of transmitters of at least FIGS. 2, 7, 8 and 9 may include at least one device 360 and/or at least one component of device 360. As shown in FIG. 3B, device 360 may include bus 362, processor 364, memory 366, storage component 368, input component 370, output component 372, and communication interface 214.

Bus 362 may include a component that permits communication among the components of device 360. In some non-limiting embodiments, processor 364 may be implemented in hardware, firmware, or a combination of hardware and software. For example, processor 364 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 366 may include a random access memory (RAM), a read only memory (ROM), and/or another type of dynamic or static storage device (e.g., flash memory, magnetic memory, optical memory, etc.) that stores information and/or instructions for use by processor 364.

Storage component 368 may store information and/or software related to the operation and use of device 360. For example, storage component 368 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 370 may include a component that permits device 360 to receive information, such as via user input (e.g., a touch screen display, a keyboard, a keypad, a mouse, a button, a switch, a microphone, etc.). Additionally, or alternatively, input component 370 may include a sensor for sensing information (e.g., a global positioning system (GPS) component, an accelerometer, a gyroscope, an actuator, etc.). Output component 372 may include a component that provides output information from device 360 (e.g., a display, a speaker, one or more light-emitting diodes (LEDs), etc.).

Communication interface 374 may include a transceiver-like component (e.g., a transceiver, a separate receiver and transmitter, etc.) that enables device 360 to communicate with other devices, such as via a wired connection, a wireless connection, or a combination of wired and wireless connections. Communication interface 374 may permit device 360 to receive information from another device and/or provide information to another device. For example, communication interface 374 may include an Ethernet interface, an optical interface, a coaxial interface, an infrared interface, a radio frequency (RF) interface, a universal serial bus (USB) interface, a Wi-Fi interface, a cellular network interface, and/or the like.

Device 360 may perform one or more processes described herein. Device 360 may perform these processes based on processor 364 executing software instructions stored by a computer-readable medium, such as memory 366 and/or storage component 368. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 366 and/or storage component 368 from another computer-readable medium or from another device via communication interface 374. When executed, software instructions stored in memory 366 and/or storage component 368 may cause processor 364 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software.

The number and arrangement of components shown in FIG. 3B are provided as an example. In some non-limiting embodiments, device 360 may include additional components, fewer components, different components, or differently arranged components than those shown in FIG. 3B. Additionally, or alternatively, a set of components (e.g., one or more components) of device 360 may perform one or more functions described as being performed by another set of components of device 360.

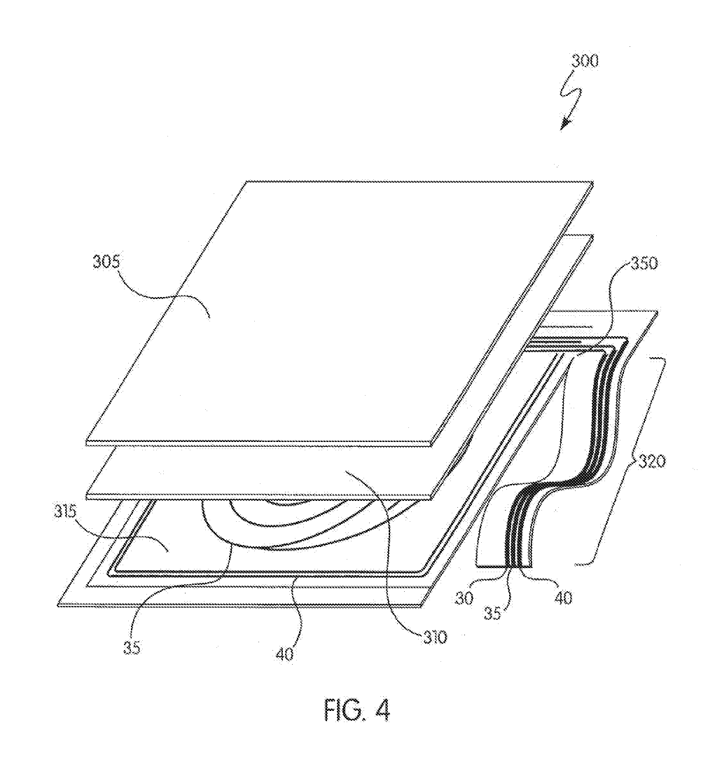

With reference to FIG. 4, the sensor pad includes a top layer 305, an absorption layer 310, and an integrated sensor layer 315. A thin topcoat on the top layer absorbs fluid quickly and has other features, such as doesn't stick to wounds easily. The top layer 305, the first layer of the pad 10, acts as a cover and is made of a flexible material.

Top layer 305 can act as a distribution layer that wicks fluid across a wider area to spread it out. Adjacent is an absorption layer 310 that ensures complete absorption and is also made of a flexible material. This absorbent core is where the fluid is ultimately stored. This layer may include a powder held between layers of absorbent fiber, and this powder forms into a gel as it absorbs fluid. The gel will not release the fluid under pressure, keeping the patient more dry.

The integrated sensor layer 315 has one or more multiple integrated sensors, 30, 35, 40 that form a circuit and are connected to the tail. Layer 315 is a waterproof layer. In one embodiment, it is formed of polypropylene onto which sensor ink is printed. Each of sensors 30, 35, 40 forms a separate circuit. The transmitter is operative to send electricity through the sensors. The one or more multiple integrated sensors 30, 35, 40 are positioned on the integrated sensor layer 310 at a specific location. The last layer is a strengthening layer, providing a final layer applied that dramatically increases the tensile strength of the pad, especially once the other layers are wet. It also has a finish that increases the friction against a bed sheet, helping to stay flat on the bed and resist wrinkling/wadding up.

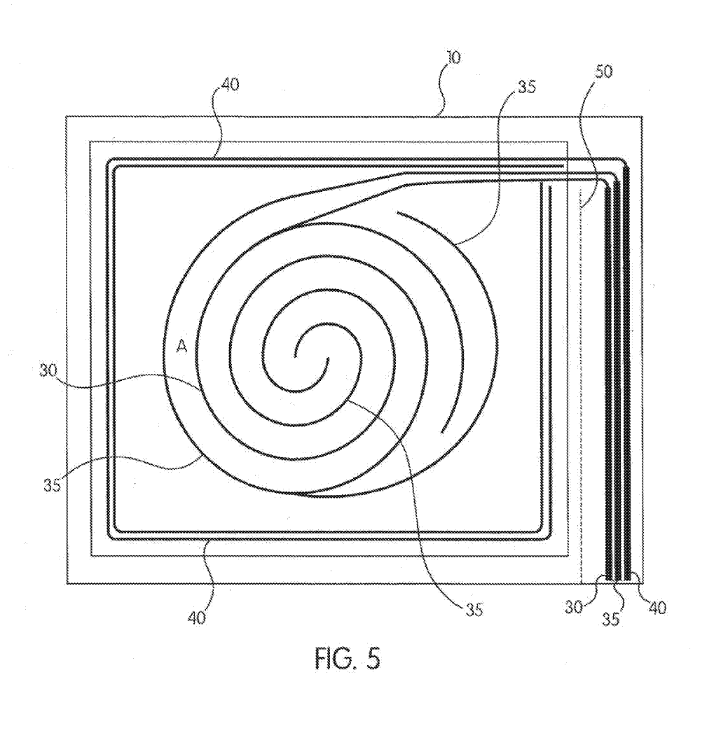

With reference to FIG. 5, the numbered sensors are arranged in a predetermined fashion to ensure fast recognition of moisture on the pad. For example, the one or more sensors are laid out on the integrated sensor layout 310 in a circular fashion and the distance between the sensors may be 2.1 inches. This circular layout begins at the lower right end of the integrated sensor layout 310 where the sensors begin to go upward to the integrated sensor layout 310 and then wrap to the left. The right sensor 40 goes outward and around the outer edge of the integrated sensor layout. The left and middle sensors, 30 and 35, go outward and curve in a circular fashion into the center of the integrated sensor layout, as shown in FIG. 5. The sensors form an interior central detection zone and a perimeter zone. The one or more sensors can alternatively be laid out on the integrated sensor layout in a different layout, i.e., in a rectangular fashion. In other embodiments, the transmitter can be modified to handle a pad sensor with other numbers of sensors, such as four or more.

The beginning of the sensors 30, 35, 40 shown in FIG. 5 occurs on one edge of the integrated sensor layout 310. In an embodiment, the edge of the integrated sensor layout 310 is perforated along one edge so that it can be ripped off from the body of the pad 10. This perforated edge with sensors 30, 35, 40 defines a tail end of the integrated sensor layout.

With continuing reference to FIG. 5, the middle sensor 35 is energized with a ground signal, such that when moisture is present on the integrated sensor layout 310, a circuit is formed and the tail end forms a coupling with the connectors of the transmitter. The transmitter operates to recognize characteristics of the electric signals in the circuits formed in the sensor pad.

In an exemplary embodiment, each of the layers of the multi-layer location-based sensor pad may be made of an absorbent material. The sensor pad may be placed on a flat surface (e.g., a patient bed, a patient chair) and may also be placed on surfaces not flat, where the pad can take the shape of the surface. The pad can also be wrapped around a patient's body or configured to provide sufficient coverage for incontinence detection. The sensor pad may be placed inside a wearable unit and may take the shape of the wearable unit. In one exemplary embodiment, a sensor and/or sensor pad may be attached to an interior of a garment. For example, a sensor may be attached to an interior of a garment such as, for example, briefs, diapers, pull-ups, or other wearable garments. In such embodiments, a sensor may be printed directly into a wearable garment with a tail coming out of a portion of the garment to facilitate the attachment with a transmitter.

As shown in FIG. 5, the layout pattern of the one or more sensors is used to determine where the moisture is present on the integrated sensor 40. In an embodiment, if moisture is detected between 35 and 40, at location A, a circuit will be formed there, determining that the moisture is present. In another embodiment, the transmitter determines the capacitance of the completed circuits including the sensors. In another example, the transmitter can read another physical property (such as inductance, temperature, and impedance). The transmitter may use the physical property of the particular sensors to determine the characteristics of the detected moisture (e.g., density, location, type). As an example, the sensors 35 transmit a ground current and the other sensors are always on and have an electrical current. When moisture touches the other sensors (e.g., 30 and 40), the physical properties of the sensors will change, the smart transmitter will determine the change (e.g., drop in impedance) and collect the moisture information. This moisture information may include the characteristics of the detected moisture.

In one non-limiting embodiment, the multi-layer location-based sensor can be used to detect the presence of moisture according to the transmitter detection of a change in physical property from the presence of moisture and the completion of the circuit on the sensor. The moisture is detected when it absorbs down through each of the layers of the pad onto the sensor. As an example, if moisture is applied to the top right portion of the top layer 300, it may be absorbed through the top layer 300 and down into the top right portion of the absorption layer 305. The moisture may then be absorbed through the top right portion of the absorption layer 305 and into the top right portion of the integrated sensor layer 310 (e.g., onto the sensors in the top right portion 35, 40). The sensors 35, 40 in the top right portion will indicate moisture on the integrated sensor layer 310, which may then correspond to the tail end. The smart transmitter may determine from the tail end the presence of moisture related to the sensors in the top right portion (e.g., 35, 40).

With continuing reference to FIG. 5, the connectors 230, 235, 240 have conductive pins that are inserted into the sensors 30, 35, 40 of the tail portion of the sensor pad. The tail is created by separating the tail from the sensor pad body along line 50. By pulling the sections apart, the tail is liberated from the sensor pad body and free to move in a more flexible manner.

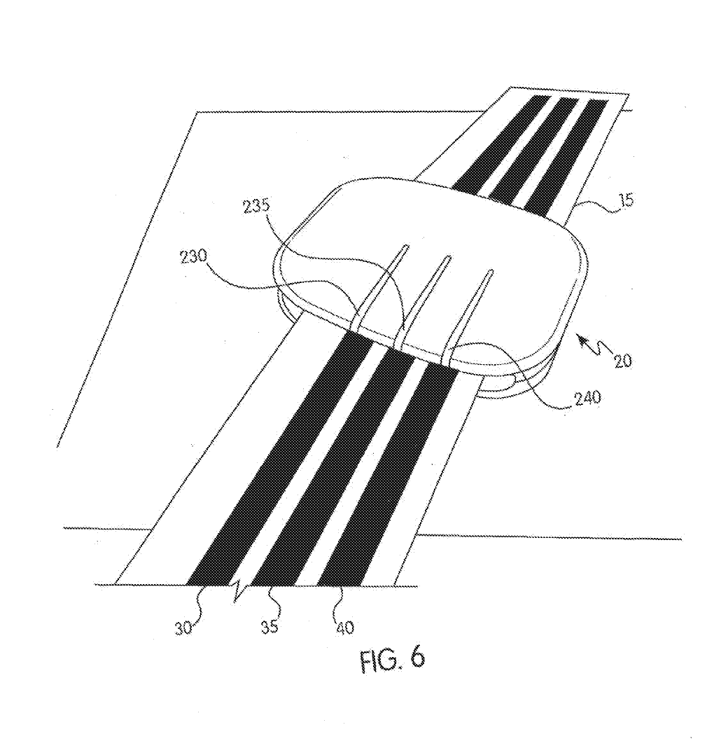

With reference to FIG. 6, the smart transmitter 20 is clamped onto the tail 15 so that the tail 15 can pass through the cavity 205 and provide aligning physical connectivity for the connectors 230, 235, 240, with the sensors, 30, 35, 40, on the tail 15. In FIG. 6, the tail portion 320 is shown partially separated from the sensor pad body, with a partial connection 350 formed, after the partially detachment or separation of the tail, forming a small bridge connection 350 which holds the sensors 30, 35, 40 in place while the pad is being used. This bridge connection can be torn when the pad is removed. When the pad is being replaced, the tail can be completely separated by pulling the tail from the body, separating and disconnecting the sensors that are bridged across that line. This tail portion provides flexibility for replacement, because the dirty pad can quickly and easily be dispelled. Also, the perforation can have alternative configurations, for example one of skill in the art could envision a perforated line extending only halfway, where the tail does not pull off, but stays attached. When changing, instead of pulling the tail off and disposing of the pad, the tail would first be removed from the transmitter and then the entire sensor pad, both body and tail, could be disposed together. The connectors 230, 235, 240 on the transmitter 20 provide the connection to the sensors 30, 35, 40 on the tail 15. The smart transmitter may also include a T-connector 245, which may be perpendicularly connected to connector 235 and lined up with sensor 35. The connectors are electrically coupled individually to the sensors such that moisture information may be transmitted from the sensors to the connectors. The connectors 230, 235, 240, 245 made of a conductive material are electrically coupled together in multiple ways including the receiver sticking into the sensors, the receiver going up against the sensor, or any combination thereof. In an embodiment, the connectors 230, 235, 240 have conductive pins that are inserted into the sensors 30, 35, 40 to create the electrical connection. Through use of the connection, the smart transmitter 20 receives the moisture information from the sensors 30, 35, 40 to determine when and where the moisture is present on the integrated sensor layer 310.

The microprocessor 102 controls the current and/or voltage to a sensor. The microprocessor 102 provides voltage across the sensors to determine if a circuit is present as a result of the presence of moisture. The initial physical property of the sensors is determined and stored and then when moisture is present, the physical property will change and alert the microprocessor 102, which will gather the sensor information. For example, the resting sensors have a certain physical property or capacitance. Thus, when moisture is present, the circuit is completed and the capacitance changes, which the transmitter 20 will detect and record.

The microprocessor 102 processes instructions on the memory, including an algorithm, for determining the original physical property of a sensor and storing the physical properties in memory. The microprocessor 102 is always on but could be programmed to use a clock cycle, for example, a clock placed on the board and coupled to the microprocessor, configured to wake up in response to receiving a notification from the sensor layout that moisture is present. The transmitter receives the moisture information and can process received information to manipulate and modify it (e.g., analyze, categorize, calculate, convert). The microprocessor 102 may store the moisture information and modify it over time. The microprocessor 102 can be connected to a radio in the smart transmitter 20 such that the transceiver 106 receives the modified information from the microprocessor 102 and may send the information to a processing device 25. The messages can be sent wirelessly.

The transceiver 106 sends signals or messages to a network, a computer, other transmitters, or any other device configured to receive and operate on the transmitted signals. The signals are sent in messages and can communicate information about the pad and patient using the pad. Zigbee, Bluetooth, or proprietary formulation may be used for communication. The transmitter sends data when the status of the pad changes (dry to wet, disconnected, etc.) as well as a ‘heartbeat’ so that we know it's still on the network. The information can include that the pad 10 is wet, where on the pad is wet, or the saturation level, and information about the location, and the name of the patient associated with a particular pad. The network can modify the information. The third-party device 25 can use the signals or messages and can display them so that a user can react to them. Continence data includes information about the patient's toileting, consisting of urine levels, fluid and diet nutrition levels during time periods, time that the resident passes urine, type and volume of drinks, degree of wetness, number of pad changes, length of time exposed to soiled environment, number of clothing and/or bedding changes, medical circumstances, type of bowel movement, time of bowel movement, day of bowel movement, Bristol stool scale classification, constipation data, whether a catheter is in place, and risk of fall while attempting to toilet.

As an example, a care facility employee will place pad 10 on top of a bed with the tail end hanging off. The care facility employee will then take a transmitter 20 and attach it to the tail end, such that it is securely fastened to the tail end and electrically coupled to the sensors on the tail. The transmitter 20 will be turned on such that the middle sensor will be on and supplying the pad 10 with power. Once the pad 10 is saturated, the transmitter will read the pad 10, send the signals to the network which will send the signals to a third-party device 25, alerting a care facility employee to come and change the sheet. The transmitter 20 and the connected tail end can be ripped off of the pad 10 by using the perforation such that the transmitter 20 and tail end are preserved. Further, the sheet and pad can easily be cleaned up.

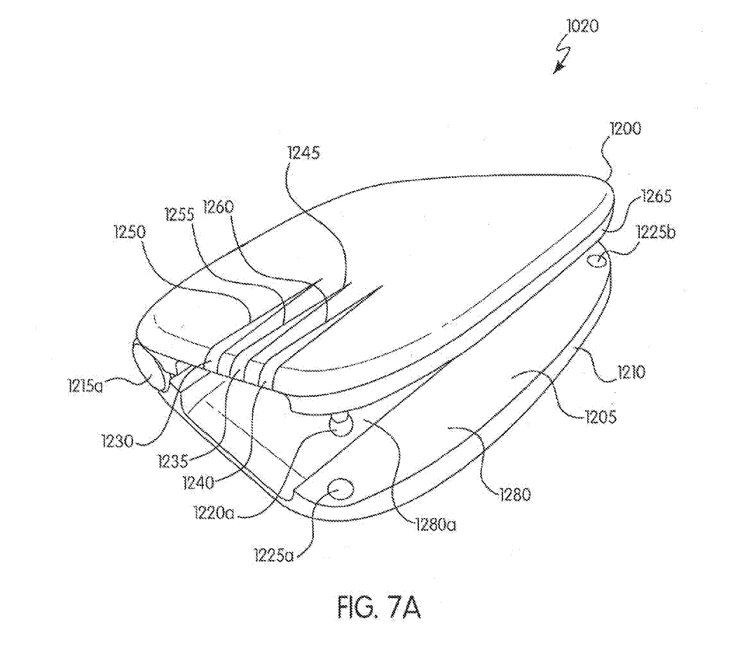

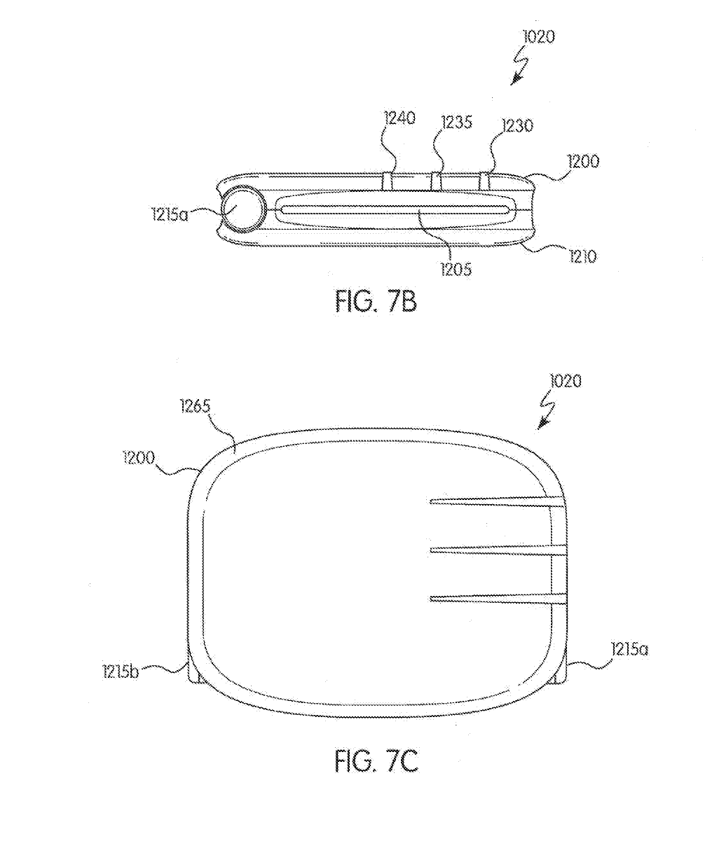

With reference to FIG. 7A, the transmitter 1020 has a top panel 1200 and a bottom panel 1210 that are connectably secured together from the side by a movable connecting piece 1215a, 1215b forming a clamshell with congruent joints on either end. The movable connecting pieces 1215a, 1215b form an axis of rotation about the joint, where the panels can move about the axis, to open and close, by rotating the panels 1200 and 1210, relative to each other. An internally curved inside surface is formed, such that when the pieces are closed together a cavity 1205 is formed. The internally curved insides are stepped internally down such that an inner portion 1280 is thinner than the outer portion. Extending outward from the top panel 1200 is a male connector 1220a on one side and male connector 1220b on the other side. Extending outward from bottom piece 1210, and directly opposite the male connectors of top panel 1200, are female openings 1225a and 1225b. The male connectors 1220a, 1220b form a locking connection between the panels 1200 and 1210 when they are inserted into the mating female openings 1225a, 1225b and received therein. However, one of ordinary skill would recognize that locking surfaces can be formed with other means, where the transmitter may be closed and secured.

With reference to FIGS. 7B and 7C, the transmitter 1020 of FIG. 7A is shown from various angles. With reference to FIG. 7B, the transmitter 1020, when closed, is secured by inserting male connectors 1220a, 1220b into the female connectors 1225a, 1225b, respectively. The closing of the transmitter 1020 will define cavity 1205 through the closed transmitter 20. The cavity 1205 provides an opening for the tail, that is to be inserted, or has already been inserted, into the transmitter 1020 and coupled to the transmitter 1020. With reference to FIG. 7C, the transmitter 1020 is shown from the top with the curved edges of the top piece 1200 rounded to prevent unintended contact or puncture of the sensor material. The rounded surfaces are also adapted to fit into a care givers hand and facilitate quickly opening and closing. In one embodiment, the transmitter can have a locking mechanism as shown. In addition, the connector can be adapted to lock and open by closing and pressing to lock, or pressing to unlock and open.

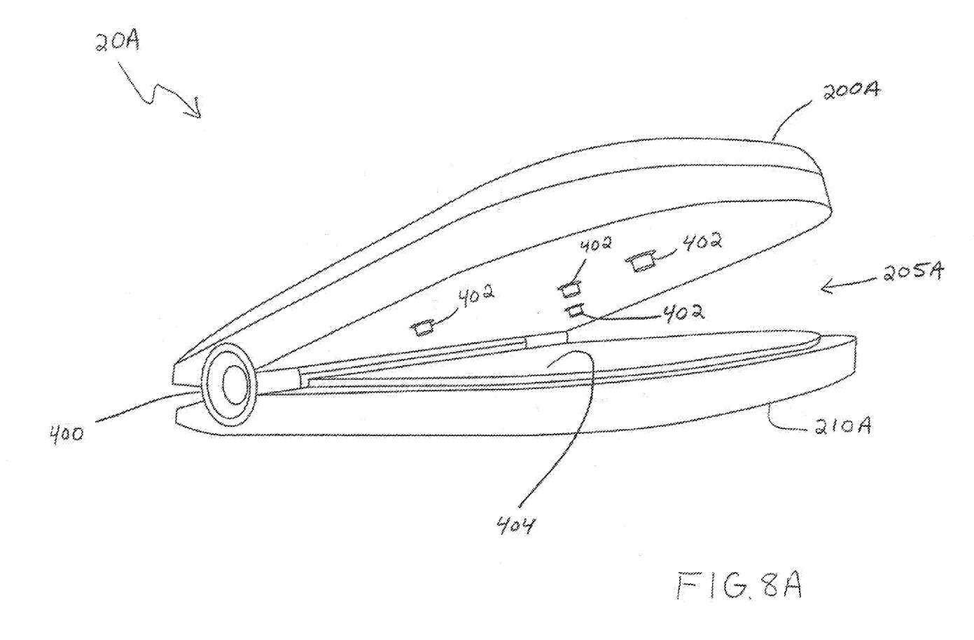

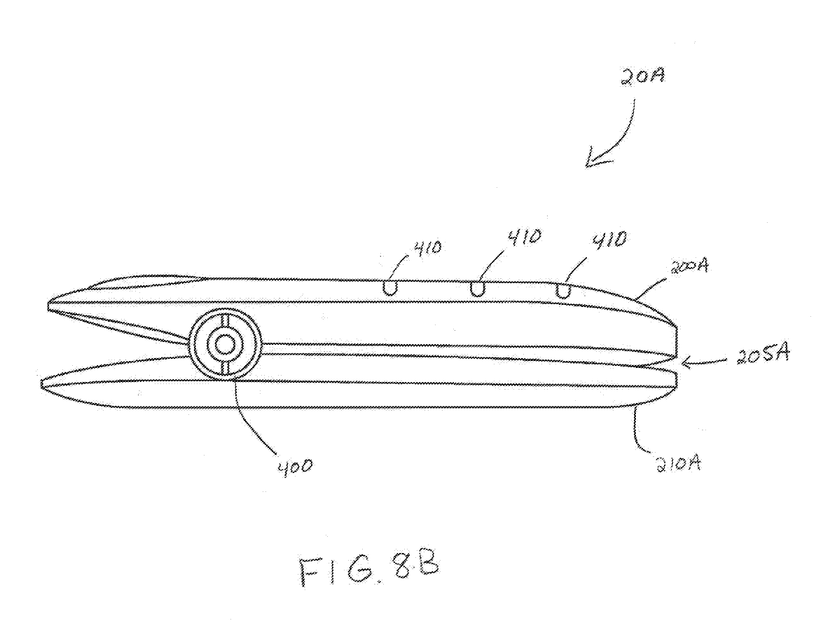

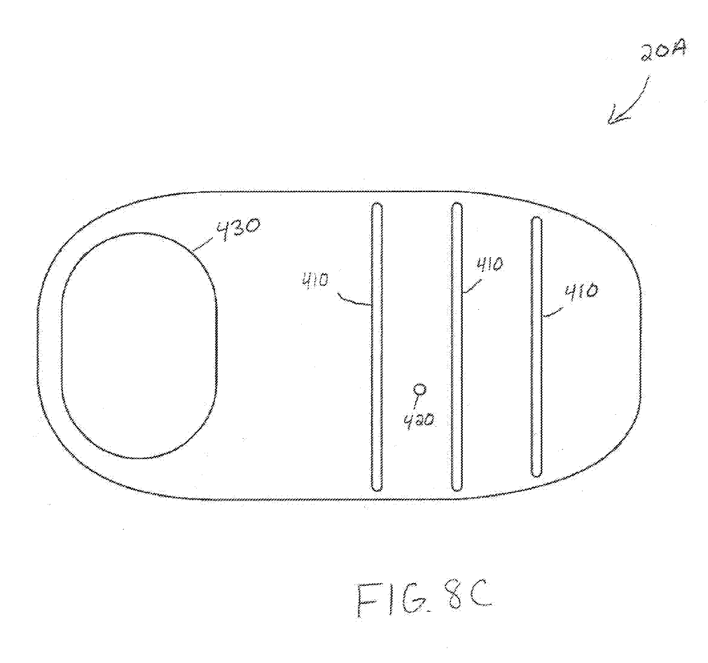

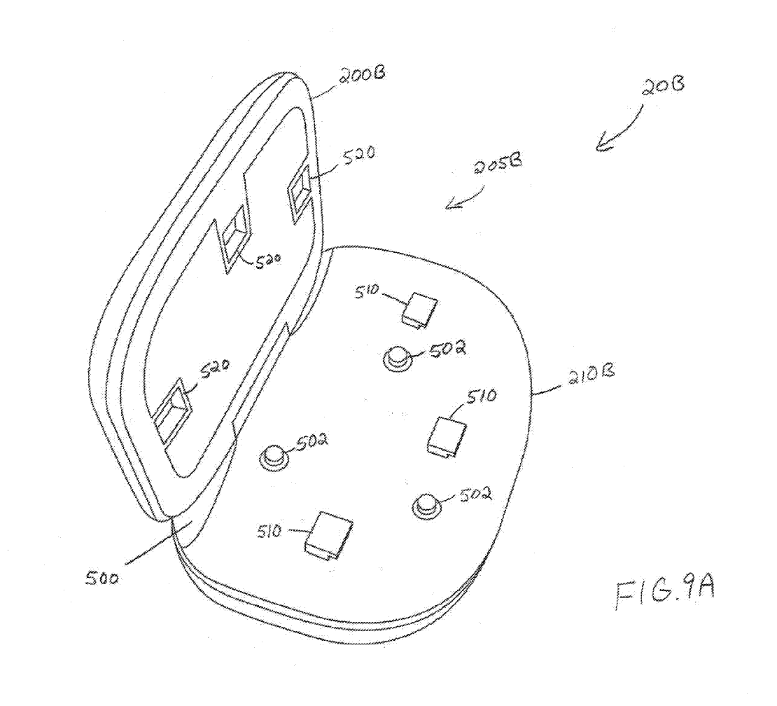

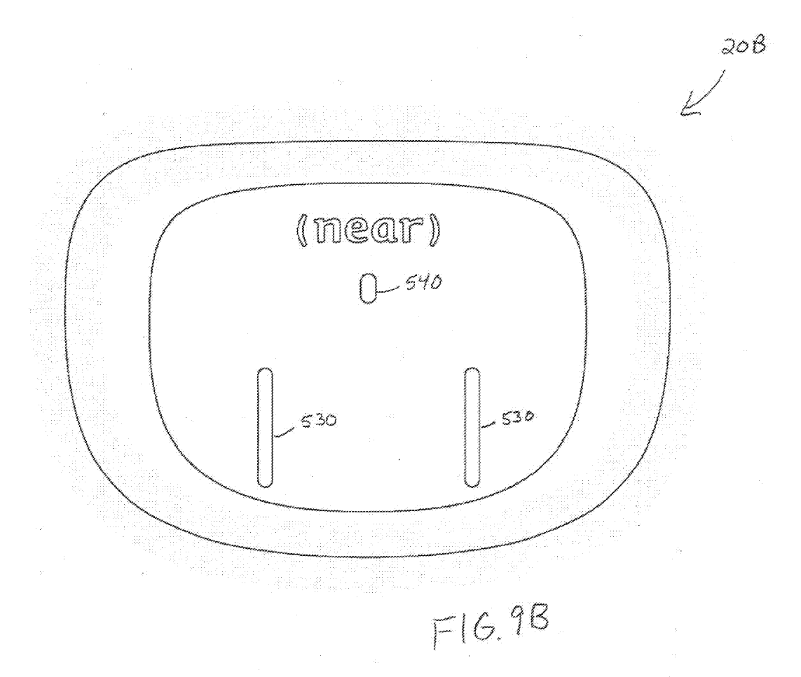

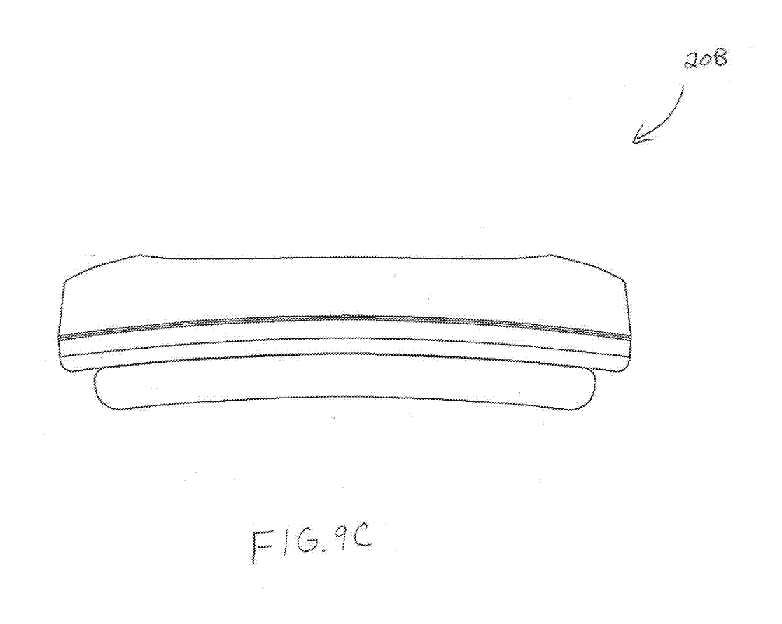

FIGS. 8A-9C illustrate other exemplary embodiments of transmitters of the present disclosure. The embodiment illustrated in FIGS. 8A-8C includes similar components to the embodiment illustrated in FIGS. 2A-2G, and the similar components are denoted by a reference number followed by the letter A. The embodiment illustrated in FIGS. 9A-9C includes similar components to the embodiment illustrated in FIGS. 2A-2G, and the similar components are denoted by a reference number followed by the letter B. For the sake of brevity, these similar components and the similar steps of using transmitter 20A (FIGS. 8A-8C) and transmitter 20B (FIGS. 9A-9C) will not all be discussed in conjunction with the embodiments illustrated in FIGS. 8A-8C and FIGS. 9A-9C.

Referring to FIG. 8A, in one exemplary embodiment, a transmitter 20A generally includes a printed circuit board, a power supply enclosed in a plastic shell having a top portion 200A and a bottom portion 210A, a movable connecting piece 400, e.g., a side hinge, and pins 402 attached to the printed circuit board. In one exemplary embodiment, the transmitter 20A includes four pins 402. In other exemplary embodiments, other number of pins 402 may be utilized. In one embodiment, the pins 402 extend through the casing to form the connection points to both power and receive a signal from the sensor. Although in FIG. 8A the pins 402 appear to have a flat head, it is contemplated that the heads of the pins 402 have teeth. For example, in one embodiment, these pins 402 are crowned, e.g., the heads of the pins 402 have teeth allowing them to reliably penetrate through a top layer of non-woven textile on the tail and penetrate into the sensor ink. The plastic casing is spring hinged on one side of the transmitter 20A allowing it be operated with one hand to easily close around the tail element of the pad. The bottom portion 210A of the transmitter 20A includes a rubber backing 404 applied to it for the pins 402 to lightly sink into.

Referring to FIG. 8B, in one exemplary embodiment, the transmitter 20A is shown from a side to illustrate a portion of guide lines 410 to be used to line up with the sensor trances. Referring to FIG. 8C, in one exemplary embodiment, the transmitter 20A is shown from the top to illustrate the guide lines 410. The transmitter 20A also includes an indicator LED 420 and a rubberized grip pad 430 for easier operation of the spring hinge.

Referring to FIG. 9A, in one exemplary embodiment, a transmitter 20B includes pins 502 and functions generally the same as transmitter 20A but the movable connecting piece 500 that movably connects the top portion 200B and the bottom portion 210B is not spring tensioned. The movable connecting piece 500, a male connection clip portion 510, and a female connection clip portion 520 form a locking mechanism to keep the transmitter 20B affixed to a disposable garment. This locking mechanism is also tamper proof and requires a non-obvious application of directional force to release so that patients will not play with or inadvertently remove the transmitter 20B.

Referring to FIG. 9B, in one exemplary embodiment, the transmitter 20B is shown from a front side to illustrate the guide lines 530 to attach to a sensor. In one embodiment, a brief only has two trace lines and is hit by three pins, e.g., two ground and an open. The transmitter 20B also includes an LED 540. Referring to FIG. 9C, in one exemplary embodiment, the transmitter 20B is shown from a bottom side to illustrate the transmitter 20B when it is closed or locked position with all the male clip portions 510 and female clip portions 520 connected up.

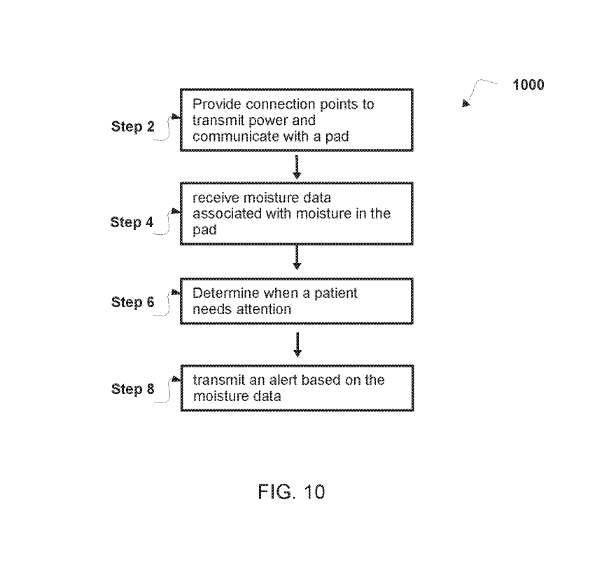

Referring to FIG. 10, In some non-limiting embodiments, a computer-implemented monitoring method 1000 includes steps for patient care using in a patient incontinence monitoring system. At step 2, the monitoring method 100 includes providing connection points to transmit power and communicate with a pad. For example, monitoring method 100 provides the connections points for transmitting power to a sensor pad and/or communicating based on one or more signals, sensor pad data (e.g., moisture data) associated with the sensor pad.

In some non-limiting embodiments, at step 4, the monitoring method 100 includes, receiving and/or transmitting moisture data associated with moisture in the pad. For example, moisture data may be transmitted or received from a transmitter, from a sensor pad, from a device coupled to the transmitter, or from a central computer system associated with the monitoring method such as patient monitoring system or other third party patient care systems.

In some non-limiting embodiments, at step 6, the monitoring method 100 includes determining when a patient needs attention. For example, monitoring system includes determining when a sensor pad associated with a patient has moisture. In some aspects, the monitoring method determines when a sensor pad associated with a patient meets a threshold of moisture in the pad.

In some non-limiting embodiments, at step 8, the monitoring method 100 includes transmitting an alert based on the moisture data. For example, an alert may be transmitted to a patient care system, for automatically updating a patient care worker that a patient needs a bed change. In some non-limiting embodiments, an alert may be based at least partially on data from patient care system. In some non-limiting embodiments, the data from a patient care system may include historic data associated with a patient sensor pad.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A monitoring device, comprising:
a sensor configured to determine moisture data associated with moisture in a pad; and
a transmitter configured to connect to the sensor and transmit the moisture data to a computer system comprising one or more processors.

2. The monitoring device of claim 1, wherein the moisture data is associated with at least one of the following: a moisture level in the pad, a moisture location in the pad, a moisture type in the pad, or any combination thereof.

3. The monitoring device of claim 1, wherein the sensor is attached to an interior of a garment.

4. The monitoring device of claim 1, wherein the sensor and the transmitter are removably connectable.

5. The monitoring device of claim 1, further comprising a sensor pad comprising:
a top layer formed of a flexible material;
an integrated sensor layer including the sensor; and
an absorption layer disposed between the top layer and the integrated sensor layer.

6. The monitoring device of claim 5, wherein the top layer wicks a fluid across an area of the top layer.

7. The monitoring device of claim 5, wherein the absorption layer absorbs and stores a fluid.

8. The monitoring device of claim 5, further comprising a powder disposed within the absorption layer, wherein the powder forms into a gel as the absorption layer absorbs a fluid.

9. The monitoring device of claim 5, wherein the integrated sensor layer further comprises:
a first sensor arranged in an interior central detection zone; and
a second sensor arranged in a perimeter detection zone.

10. The monitoring device of claim 5, wherein the integrated sensor layer further comprises:
a first sensor arranged in a first spiral configuration;
a second sensor arranged in a second spiral configuration; and
a third sensor arranged in a rectangular configuration outside the first spiral configuration and the second spiral configuration.

11. The monitoring device of claim 5, wherein the integrated sensor layer is waterproof.

12. The monitoring device of claim 5, wherein a portion of the integrated sensor layer includes a perforation.

13. The monitoring device of claim 5, wherein the transmitter comprises:
a top portion;
a bottom portion; and
a connection portion that movably connects the top portion and the bottom portion between an open position and a closed position.

14. The monitoring device of claim 13, wherein, with the connection portion in the closed position, a cavity is formed between the top portion and the bottom portion.

15. The monitoring device of claim 14, wherein the transmitter further comprises an electrical connector.

16. The monitoring device of claim 15, wherein, with the connection portion in the closed position, a portion of the sensor is received through the cavity of the transmitter and contacts the electrical connector to connect the transmitter and the sensor.

17. The monitoring device of claim 16, wherein the sensor is energized with a ground signal, and wherein, with moisture on the integrated sensor layer, a circuit is formed with the transmitter.

18. The monitoring device of claim 17, wherein the transmitter comprises at least one processor programmed or configured to recognize and transmit characteristics of electric signals in the circuit.

19. The monitoring device of claim 17, wherein the transmitter comprises at least one processor programmed or configured to monitor, with the computer system, the moisture information and send an alert to a user based on the moisture information.

20. The monitoring device of claim 18, wherein the processor is programmed or configured to recognize and transmit a capacitance of the sensor.

21. The monitoring device of claim 18, wherein the processor is programmed or configured to recognize and transmit an inductance of the sensor.

22. The monitoring device of claim 18, wherein the processor is programmed or configured to recognize and transmit a temperature of the sensor.

23. The monitoring device of claim 18, wherein the processor is programmed or configured to recognize and transmit an impedance of the sensor.

24. The monitoring device of claim 18, wherein the processor is programmed or configured to recognize and transmit characteristics of the moisture information.

25. The monitoring device of claim 13, wherein the transmitter is re-usable and the sensor is disposable.

26. A monitoring system, comprising:
a sensor configured to determine moisture data associated with moisture in a pad;
a transmitter configured to connect to the sensor and transmit the moisture data to a computer system comprising one or more processors; and
the computer system configured to receive the moisture data from the transmitter to determine when a patient needs attention and send an alert to a user based on the moisture data.

27. A computer-implemented method for monitoring a pad, comprising:
providing connection points, the connections points for transmitting power to, and receiving one or more signals associated with, a pad;
receiving, with a computer comprising one or more processors, moisture data associated with moisture in the pad;
determining, with a computer comprising one or more processors, when a patient needs attention; and
transmitting, with the computer comprising one or more processors, an alert based on the moisture data.
